(19) United States
(12) Patent Application Publication
(10) Pub. No.: US 2016/0278645 A1
(43) Pub. Date: Sep. 29, 2016

(54) APPARATUS AND METHOD FOR DETECTING BIOMETRIC INFORMATION OF A LIVING BODY

(71) Applicant: SAMSUNG ELECTRONICS CO., LTD., Suwon-si (KR)

(72) Inventor: Youngzoon YOON, Hwaseong-si (KR)

(73) Assignee: SAMSUNG ELECTRONICS CO., LTD., Suwon-si (KR)

(21) Appl. No.: 14/862,288

(22) Filed: Sep. 23, 2015

(30) Foreign Application Priority Data
Mar. 25, 2015 (KR) 10-2015-0041643

(51) Int. Cl. A61B 5/021; A61B 5/00

(52) U.S. Cl. CPC A61B 5/02141; A61B 5/721; A61B 5/7278; A61B 5/02125; A61B 5/681; A61B 2090/306

(57) ABSTRACT

An apparatus for detecting biometric information includes a photo sensor configured to acquire a first signal by detecting a change in an optical signal with respect to an object; a pressure sensor configured to acquire a second signal by detecting a change of a pressure with respect to the object; a signal extractor configured to extract a waveform signal from at least one from among the first signal and the second signal based on signal sensitivities of the first and second signals; and a signal analyzer configured to analyze biometric information from the extracted waveform signal.

### CROSS-REFERENCE TO RELATED APPLICATION

This application claims priority from Korean Patent Application No. 10-2015-0041643, filed on Mar. 25, 2015, in the Korean Intellectual Property Office, the disclosure of which is incorporated herein in its entirety by reference.

### BACKGROUND

1. Field

Apparatuses and methods consistent with exemplary embodiments relate to detecting biometric information of a living body.

2. Description of the Related Art

Blood pressure is measured to assess health of an individual, and a sphygmomanometer, which is a device for measuring blood pressure, is commonly used in medical institutions and at home. A cuff-based sphygmomanometer measures a systolic blood pressure and a diastolic blood pressure while a region through which an artery passes is pressed via a cuff such that the blood flow through the artery is stopped and the pressure is slowly reduced.

Although the cuff-based sphygmomanometer may accurately measure blood pressure, it has a large volume and is inconvenient to carry and is thus not suitable as a wearable device and for real-time monitoring of a continuous change in blood pressure. Therefore, much research on a cuffless sphygmomanometer has been recently conducted.

A cuffless method of measuring blood pressure may include a pulse transit time (PTT) method in which the blood pressure is measured by using a correlation of the blood pressure with a time difference between electrocardiography (ECG) and photoplethysmography (PPG). As the PPT method uses ECG, the PPT method is not suitable for a continuous measurement of the blood pressure using a single band.

Methods for detecting biometric information of an individual such as pulse waves may be largely classified into invasive methods and noninvasive methods. The noninvasive methods are capable of simply detecting pulse waves without causing pain of a patient and are usually used in conjunction with wearable devices.

For an accurate pulse wave analysis (PWA), optical or pressure signal-based information of a certain surface area of an object to be diagnosed is needed. Biometric information of an object may be obtained on the basis of optical or pressure signal-based information and various methods are used to reduce measurement errors of the biometric information.

### SUMMARY

One or more exemplary embodiments provide biometric information detection apparatuses and methods for continuously measuring biometric information in a contactless or contact manner.

Additional aspects will be set forth in part in the description which follows and, in part, will be apparent from the description, or may be learned by practice of the presented exemplary embodiments.

According to an aspect of an exemplary embodiment, provided is an apparatus for detecting biometric information, the apparatus including: a photo sensor configured to acquire a first signal by detecting a change in an optical signal with respect to an object; a pressure sensor configured to acquire a second signal by detecting a change of a pressure with respect to the object; a signal corrector configured to correct a waveform inversion that occurs in the first signal; a signal extractor configured to extract a waveform signal from at least one from among the first signal and the second signal based on signal sensitivities of the first and second signals; and a signal analyzer configured to analyze biometric information from the extracted waveform signal.

The photo sensor may be further configured to acquire the first signal in a contactless state with respect to the object or when a contact pressure with respect to the object is less than a reference value, and the pressure sensor may be configured to acquire the second signal in a contact state with respect to the object.

The photo sensor may include: a light-emitting unit including at least one light-emitting device, and is configured to emit light; and a light-receiving unit including at least one light-receiving device configured to detect the change in the optical signal.

The light-emitting unit may include a plurality of light-emitting devices that are arranged in at least one line or the light-emitting unit includes a light guide configured to guide light incident from the at least one light-emitting device.

The light-receiving unit includes a plurality of light-receiving devices, the plurality of light-receiving devices being arranged in an array along at least one side of the light-emitting unit or arranged between the plurality of light-emitting devices such that the plurality of light-receiving devices surround each of the plurality of light-emitting devices.

The light guide may have a reflective surface, on which the light incident from the at least one light-emitting device is reflected, and a translucent surface that faces the reflective surface.

The light guide may be bendable.

The photo sensor may have a structure in which a unit of a light-emitting device and a pair of light-receiving devices located at both sides of the light-emitting device is arranged in an array or the photo sensor may have a structure in which a unit of the light-emitting device and a plurality of light-receiving devices surrounding the light-emitting device is arranged in an array.

The pressure sensor may include a plurality of pressure sensors that are arranged in an array at both sides of the photo sensor in a one-to-one correspondence with the plurality of light-receiving devices.

The pressure sensor may include a plurality of pressure sensors that are arranged in an array along at least one side of the array of the plurality of light-receiving devices.

The photo sensor may include: a light-emitting unit including at least one light-emitting device, and configured to emit light; and a light-receiving unit including a plurality of light-receiving devices that are arranged in an array along at least one side of the light-emitting unit, and the pressure sensor includes a plurality of pressure sensors arranged along the array of the plurality of light-receiving devices.

The photo sensor may include a plurality of light-emitting devices that are arranged to form at least one line or the photo sensor may include a light guide configured to guide light from the at least one light-emitting device.

The light guide may be bendable.

The light-emitting device may include a laser device.

The apparatus may further include a signal corrector configured to correct a waveform inversion that occurs in the first signal, wherein the signal corrector is further configured to correct the waveform inversion that occurs in the first signal based on comparison between first-order differential values of the first signal.

The biometric information may include information about blood pressure of the object.

According to an aspect of another exemplary embodiment, provided is a method of detecting biometric information, the method including: acquiring a first signal by detecting a change in an optical signal with respect to an object; acquiring a second signal by detecting a change of a pressure with respect to the object; correcting waveform inversion that occurs in the first signal; extracting a waveform signal from at least one from among the first signal and the second signal based on signal sensitivities of the first and second signals; and analyzing biometric information from the extracted waveform signal.

The extracting may include, when a contact pressure with respect to the object is less than a reference value, extracting the waveform signal from the first signal.

The method may further include correcting waveform inversion that occurs in the first signal, wherein the waveform inversion is corrected based on comparison between first-order differential values of the first signal.

The biometric information may include information about blood pressure of the object.

### BRIEF DESCRIPTION OF THE DRAWINGS

The above and/or other aspects will become more apparent by describing certain exemplary embodiments with reference to the accompanying drawings in which:

FIG. 1 illustrates a block diagram of an apparatus for detecting biometric information according to an exemplary embodiment;

FIG. 2 illustrates a sensor applicable to the apparatus of FIG. 1, according to an exemplary embodiment;

FIG. 3 illustrates a photo sensor in the sensor of FIG. 2, according to an exemplary embodiment;

FIG. 4 illustrates a photo sensor in the sensor of FIG. 2, according to another exemplary embodiment;

FIG. 5 illustrates a photo sensor applicable to the sensor of FIG. 2, according to another exemplary embodiment;

FIG. 6A illustrates an unfolded state of a light guide in the sensor of FIG. 5, according to an exemplary embodiment;

FIG. 6B illustrates a curved state of the light guide in the sensor of FIG. 5, according to another exemplary embodiment;

FIG. 7 illustrates an arrangement relationship between a sensor including a photo sensor and a pressure sensor arranged in an array form and a blood vessel when the apparatus of FIG. 1 is worn, according to an exemplary embodiment;

FIG. 8 is a graph illustrating waveforms differently measured for each light-receiving device of a photo sensor.

FIG. 9 illustrates an arrangement in which a photo sensor and a pressure sensor are arranged in an array form on a flexible band (or strap) when the apparatus of FIG. 1 is of a band type or a watch type, according to an exemplary embodiment;

FIG. 10 shows graphs illustrating pulse waveforms measured by a photo sensor and a pressure sensor according to contactless and contact states;

FIG. 11 shows graphs illustrating error correction on measurement waveforms of a photo sensor;

FIG. 12 shows graphs illustrating comparison of a normal biometric pulse waveform (BPW), an inverse BPW, and first-order differential values thereof in measurement waveforms of a photo sensor;

FIGS. 13A to 13C illustrates graphs of various measurement waveforms for channels of a photo sensor of the apparatus of FIG. 1, according to exemplary embodiments;

FIG. 14 illustrates a flowchart of a method of detecting biometric information, according to an exemplary embodiment;

FIG. 15A illustrates a watch-type apparatus for detecting biometric information, according to another exemplary embodiment;

FIG. 15B illustrates the apparatus of FIG. 15A in a use state;

FIG. 16 illustrates a wrist band-type apparatus for detecting biometric information, according to another exemplary embodiment; and

FIG. 17 illustrates a reference diagram for describing a method of providing information on blood pressure by an apparatus for processing a biometric signal when an apparatus for detecting biometric information is implemented in a wrist band type.

### DETAILED DESCRIPTION

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects.

Hereinafter, when it is described that a certain component is “above” or “on” another component, the certain component may be directly above another component, or a third component may be interposed therebetween.

Although terms, such as ‘first’ and ‘second’, can be used to describe various elements, the elements cannot be limited by the terms. The terms can be used to classify a certain element from another exemplary element.

An expression in the singular includes an expression in the plural unless they are clearly different from each other in context. In addition, when a certain part “includes” a certain component, this indicates that the part may further include another component instead of excluding another component unless there is different disclosure.

In addition, the term, such as “ . . . unit” or “module,” disclosed in the specification indicates a unit for processing at least one function or operation, and this may be implemented by hardware, software, or a combination thereof.

As used herein, the term “and/or” includes any and all combinations of one or more of the associated listed items. Expressions such as “at least one of,” when preceding a list of elements, modify the entire list of elements and do not modify the individual elements of the list.

In the specification, the “object” indicates a target of which biometric state is to be measured, i.e., a human being, an animal, or the like. In addition, a “user” may also indicate a target of which biometric state is to be measured, i.e., the object or have a wider scope that includes a medical expert capable of using an apparatus for processing a biometric signal.

An apparatus for detecting biometric information, according to one or more exemplary embodiments, may be an apparatus that may be placed on an object, e.g., a wearable apparatus. The apparatus for detecting biometric information may be of a watch type, a bracelet type, a wrist band type, a ring type, a hairband type, or the like and may have a communication function and a data processing function. However, it is described in the exemplary embodiments below that the apparatus for detecting biometric information is a watch-type or wrist band-type apparatus, the exemplary embodiments are not limited thereto.

The apparatus for detecting biometric information, according to one or more exemplary embodiments, may measure a biometric signal in both contactless and contact states with the skin of an object with a complex sensor structure of a photo sensor and a pressure sensor, and thus the convenience of a user may be maximized, and biometric information, e.g., blood pressure, may be continuously measured. The apparatus for detecting biometric information, according to one or more exemplary embodiments, may measure an accurate blood pressure waveform by using a directional characteristic of a laser beam and a speckle characteristic due to the skin scattering of light of a single wavelength to be able to measure a blood pressure waveform in a contactless state with a photo sensor and mutually complementing, with a pressure sensor, a portion where an optical signal characteristic is lowered in a press and contact state. In addition, by implementing the complex sensor in an array structure, an accurate biometric signal waveform may be extracted by simultaneously acquiring signal waveforms from a plurality of sensors and obtaining a waveform of a maximum signal-to-ratio (SNR) of a desired blood vessel. Unlike the pressure sensor, when a waveform is measured using the photo sensor, a case where the waveform is inverted according to a skin shape and a measuring angle of the photo sensor may occurs. However, the apparatus for detecting biometric information, according to one or more exemplary embodiments, may also correct this waveform inversion phenomenon to measure an accurate waveform.

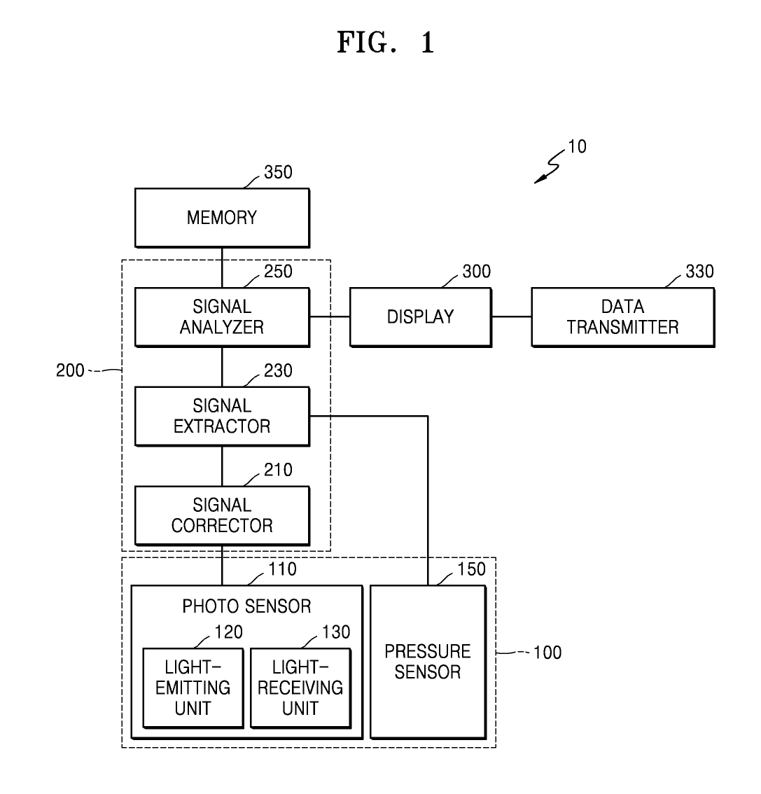

FIG. 1 illustrates a block diagram of an apparatus 10 for detecting biometric information according to an exemplary embodiment.

Referring to FIG. 1, the apparatus 10 may include a sensor 100, which may include a photo sensor 110 and a pressure sensor 150, and a signal processor 200. The apparatus 10 may further include at least one of a memory 350, a display 300, and a data transmitter 330. Hereinafter, it is described as an example that the apparatus 10 includes the memory 350, the display 300, and the data transmitter 330.

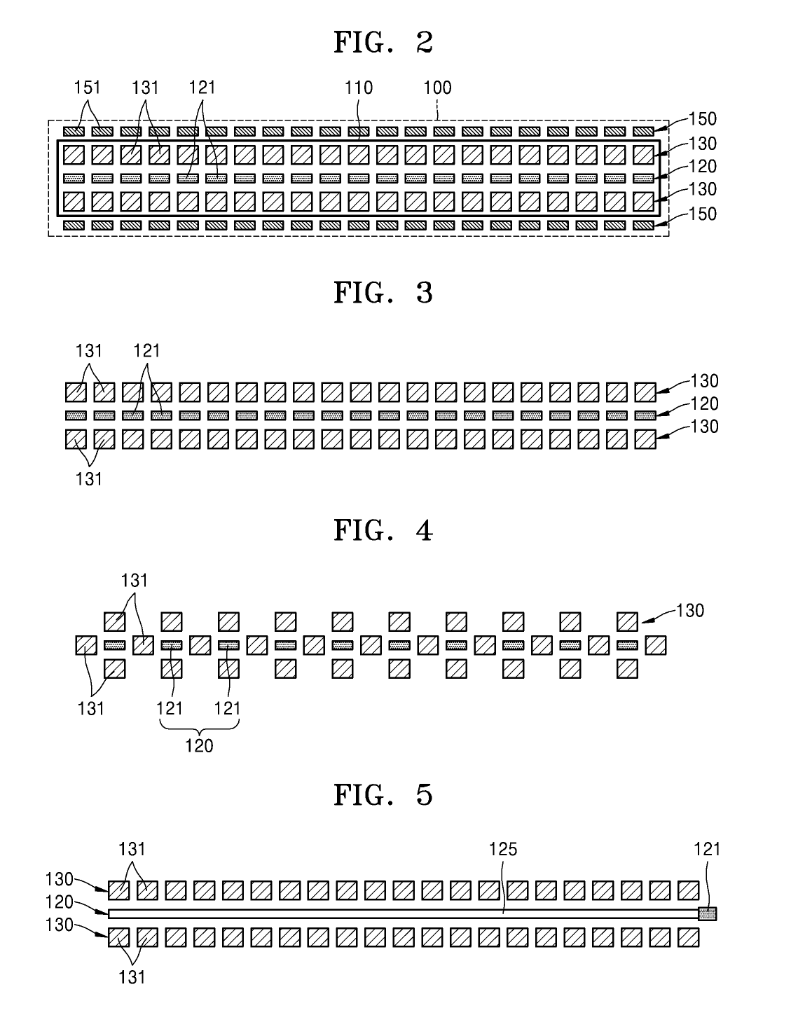

In the sensor 100, the photo sensor 110 is configured to acquire a first signal by irradiating light on an object and detecting a signal change in the light due to the object and may be configured to be able to measure a biometric signal of the object in a contactless state with the object or when a contact pressure is less than a reference value. The photo sensor 110 may include a light-emitting unit 120 and a light-receiving unit 130. The light-emitting unit 120 may be configured to include at least one light-emitting device 121 (refer to FIG. 2) and to emit light over a predetermined range. The light-receiving unit 130 may include at least one light-receiving device 131 (refer to FIG. 3) to detect an optical signal modulated by the object.

The light-emitting device 121 may be a laser device, such as a laser diode (LD), or a light-emitting diode (LED). The light-receiving device 131 may include a photo diode or an image sensor, e.g., a CMOS image sensor (CIS). For the light-receiving device 131, a photo transistor (PTr) may be used. The light-receiving device 131 may be configured to detect a signal change of light scattered or reflected from the skin or blood vessel of the object, i.e., a person to be diagnosed, according to a change in a blood flow. An arrangement of the light-emitting device 121 and the light-receiving device 131 respectively forming the light-emitting unit 120 and light-receiving unit 130 of the photo sensor 110 will be described below.

The pressure sensor 150 in the sensor 100 is configured to acquire a second signal by detecting a pressure change due to the object and may be configured to detect a biometric signal from a pressure change due to the object in a contact state with the object. That is, the pressure sensor 150 may be a contact-type pressure sensor. Arrangement of the pressure sensor 150 will be described below.

The signal processor 200 may include a signal corrector 210, a signal extractor 230, and a signal analyzer 250.

The signal corrector 210 is configured to correct waveform inversion of the first signal when the waveform inversion occurs in the first signal acquired by the photo sensor 110.

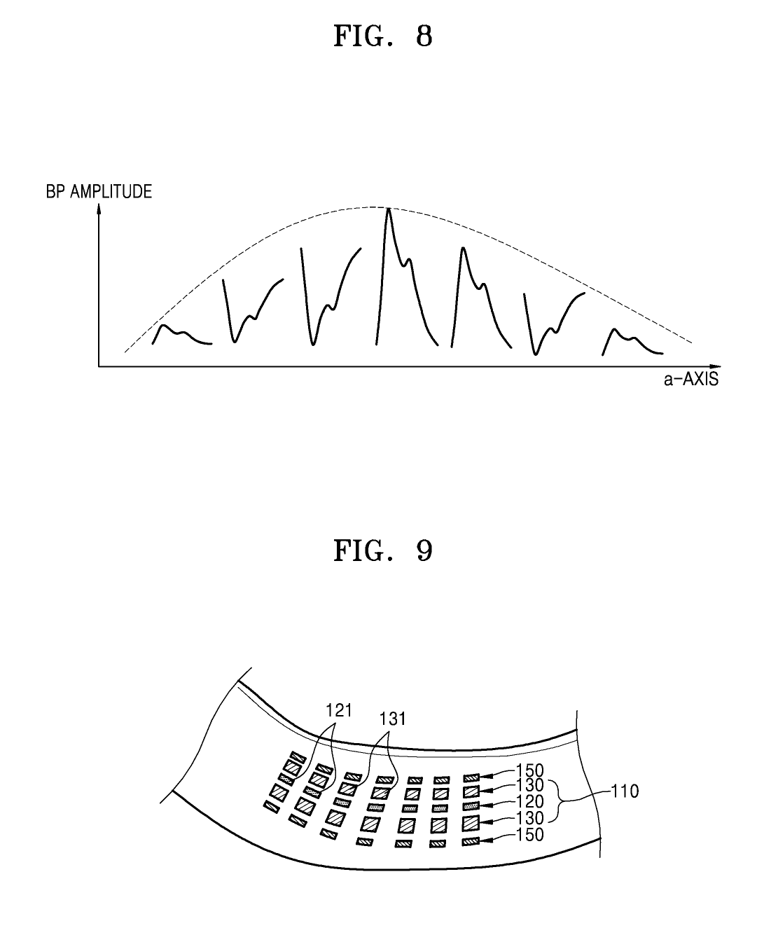

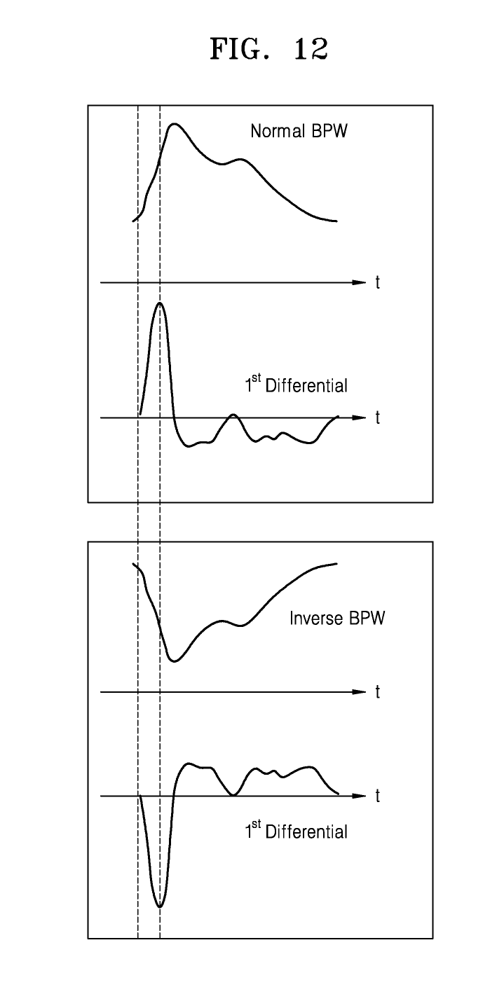

A waveform may be differently measured for each light-receiving device 131 of the photo sensor 110. That is, unlike the pressure sensor 150, when a waveform is measured using the photo sensor 110, a case where the waveform is inverted according to a skin shape and a measuring angle of the photo sensor may occurs as shown in FIG. 8. When waveform inversion of the first signal acquired by the photo sensor 110 occurs, the signal corrector 210 corrects the waveform inversion. For example, the signal corrector 210 obtains a first-order differential value by first-order differentiating the input first signal and corrects a waveform inversion error through comparison of the obtained a first-order differential value. For example, as shown in FIG. 12, when first-order differential values are obtained by first-order differentiating the first signal of a normal biometric pulse waveform (BPW) or an inverse BPW, a maximum absolute value of the first-order differential values has a positive value if waveform inversion has not occurred in the first signal, and the maximum absolute value of the first-order differential values has a negative value if the waveform inversion has occurred in the first signal. Therefore, when a waveform of the first signal of which a maximum absolute value of an obtained first-order differential value has a negative value is inverted, the waveform inversion error may be corrected, thereby obtaining the first signal having a normal waveform pattern.

The signal extractor 230 is configured to determine signal sensitivities of the first and second signals and extract a pulse waveform of a desired sensitivity, e.g., a maximum sensitivity, from the first and second signals. Herein, although it is expressed for convenience that a biometric signal detected by the photo sensor 110 is the first signal and a biometric signal detected by the pressure sensor 150 is the second signal, the first and second signals may be expressed as a biometric signal without identifying the first and second signals.

The signal analyzer 250 analyzes biometric information from the pulse waveform extracted by the signal extractor 230. The signal analyzer 250 may estimate blood pressure information and the like by analyzing a waveform characteristic of the biometric signal, e.g., a photoplethysmography (PPG) pulse wave signal. The blood pressure information analyzed by the signal analyzer 250 may include, for example, systolic blood pressure, diastolic blood pressure, a heart rate, and the like.

The blood pressure information obtained by the signal analyzer 250 may be displayed on the display 300 or the like. The display 300 may be configured to display, for example, systolic blood pressure, diastolic blood pressure, and the like and may also be configured to display a heart rate, and the like. The display 300 may be provided to the apparatus 10 as one body or correspond to a display provided to a separate device.

The memory 350 may store a reference value for a contact pressure and the like and may also store an algorithm for correcting a waveform inversion error through comparison of a first-order differential value of the first signal, an algorithm for extracting a biometric signal waveform of a desired signal sensitivity, e.g., a maximum signal sensitivity, through comparison of a waveform signal distribution value. In addition, the memory 350 may store a program for processing and controlling of the signal processor 200 and input/output data. That is, the memory 350 may store measurement results of the sensor 100 and biometric information acquired by the signal processor 200 through signal processing.

The memory 350 may include at least one type of storage medium among a flash memory type memory, a hard disk type memory, a multimedia card micro type memory, a card type memory (e.g., a secure digital (SD) or extreme digital (XD) memory or the like), a random access memory (RAM), a static RAM (SRAM), a read only memory (ROM), an electrically erasable programmable ROM (EEPROM), PROM, a magnetic memory, a magnetic disc, and an optical disc.

The data transmitter 330 is configured to transmit a result analyzed by the signal analyzer 250 to another external device. The blood pressure information analyzed or estimated by the signal analyzer 250 may be displayed on the display 200, and a blood pressure value, a heart rate value, and the like may be transmitted to an external device such as a smartphone or a computer by using the data transmitter 330, e.g., a communication device such as a Bluetooth device. By using the data transmitter 330, it may be implemented that various services are provided from a hospital side by using the data transmitter 330 to connect a device to another device or a hospital.

The external device may be not only a smartphone or a computer but also, for example, medical equipment using analyzed blood pressure information, a printer for printing a result, or a display device for displaying an analysis result. Also, the external device may be one of various devices such as a tablet personal computer (PC), a personal digital assistant (PAD), a laptop computer, a PC, and other mobile or non-mobile computing device.

The data transmitter 330 may be connected to the external device in a wired or wireless manner. For example, the data transmitter 330 may be configured to communicate with the external device by using various communication schemes such as Bluetooth, Bluetooth low energy (BLE), near-field communication (NFC), wireless local area network (WLAN), ZigBee, infrared data association (IrDA), Wi-Fi Direct (WFD), ultra wideband (UWB), Ant+, Wi-Fi, and the like.

The apparatus 10 may further include a user interface (not shown). The user interface is an interface with a user and/or the external device and may include an input unit and an output unit. The user may be a target of which blood pressure is to be measured, i.e., the object, or any human being capable of using the apparatus 10, such as a medical expert. Through the user interface, information needed to operate the apparatus 10 may be inputted, and an analyzed result may be outputted. The user interface may include, for example, a button, a connector, a keypad, a display, and the like and may further include components such as an acoustic output unit and a vibration motor.

The apparatus 10 may be configured to be portable in any one of a wearable device shape, a mobile phone shape, e.g., mobile smartphone, and a tablet device shape. That is, the apparatus 10 may be equipped in a wearable device, a mobile phone, e.g., mobile smartphone, a tablet device, or the like. In addition, the apparatus 10 may be implemented in a shape inserting a finger to measure blood pressure, e.g., a finger tongs shape.

For example, the apparatus 10 may be a wearable device. In this case, the wearable device may be of a watch type, a bracelet type, a wrist band type, a ring type, an eyeglass type, an earphone type, a headset type, a hairband type, or the like. Alternatively, only partial components of the apparatus 10, e.g., the sensor 100 and the signal processor 200, may be implemented in a device wearable by the object.

FIG. 2 illustrates the sensor 100 applicable to the apparatus 10 of FIG. 1, according to an exemplary embodiment. FIG. 3 illustrates the photo sensor 110 in the sensor 100 of FIG. 2, according to an exemplary embodiment. FIG. 4 illustrates the photo sensor 110 in the sensor 100 of FIG. 2, according to another exemplary embodiment. FIG. 5 illustrates the photo sensor 110 applicable to the sensor 100 of FIG. 2, according to another exemplary embodiment. The structures of the sensor 100 applicable to the apparatus 10, according to the exemplary embodiments, are not limited to FIGS. 2 to 5, and various structures including the photo sensor 110 and the pressure sensor 150 may be applicable.

Referring to FIG. 2, the photo sensor 110 in the sensor 100 may include the light-emitting unit 120 configured to include at least one light-emitting device 121 and to emit light over a predetermined range and the light-receiving unit 130 including at least one light-receiving device 131 to detect an optical signal modulated by the object.

Referring to FIGS. 2 and 3, the light-emitting unit 120 may be linearly arranged such that a plurality of light-emitting devices 121 form at least one line. The light-receiving unit 130 may include a plurality of light-receiving devices 131 and may be arranged such that the plurality of light-receiving devices 131 form an array along at least one side of the light-emitting unit 120. FIGS. 2 and 3 show an example in which the plurality of light-emitting devices 121 of the light-emitting unit 120 are linearly arranged to form one line and the plurality of light-receiving devices 131 are arranged to form an array along both sides of the line of the plurality of light-emitting devices 121.

As described above, the photo sensor 110 may have a structure including a unit in which a light-emitting device 121 and a pair of light-receiving devices 131 located at both sides of the light-emitting device 121 are arranged in an array.

As another example, as shown in FIG. 4, the photo sensor 110 may have a structure in which the light-receiving device 131 is disposed between the light-emitting devices 121. In this case, the structure includes a unit in which the light-emitting device 121 and a plurality of light-receiving devices 131 surrounding the one light-emitting device 121 that are arranged in an array. That is, as shown in FIG. 4, the light-emitting unit 120 of the photo sensor 110 may include a plurality of light-emitting devices 121 spaced apart from each other to emit light over a predetermined range, and the light-receiving unit 130 may include a plurality of light-receiving devices 131 disposed to surround each light-emitting device 121.

For example, the plurality of light-emitting devices 121 of the light-emitting unit 120 may be linearly arranged to be spaced apart from each other and to form at least one line, and the plurality of light-receiving devices 131 of the light-receiving unit 130 may form an array along both sides of the array of the plurality of light-emitting devices 121 arranged to form at least one line and may also be disposed between light-emitting devices 121 such that a plurality of light-receiving devices 131 are arranged to surround each light-emitting device 121, e.g., arranged in a circular shape to surround each light-emitting device 121.

In this case, the photo sensor 110 may have a structure in which a unit including one light-emitting device 121 and a plurality of light-receiving devices 131, e.g., at least three or four light-receiving devices 131, surrounding the one light-emitting device 121 is arranged in an array.

As another example, as shown in FIG. 5, the light-emitting unit 120 may have a structure including at least one light-emitting device 121 and a light guide 125 for guiding light incident from the at least one light-emitting device 121.

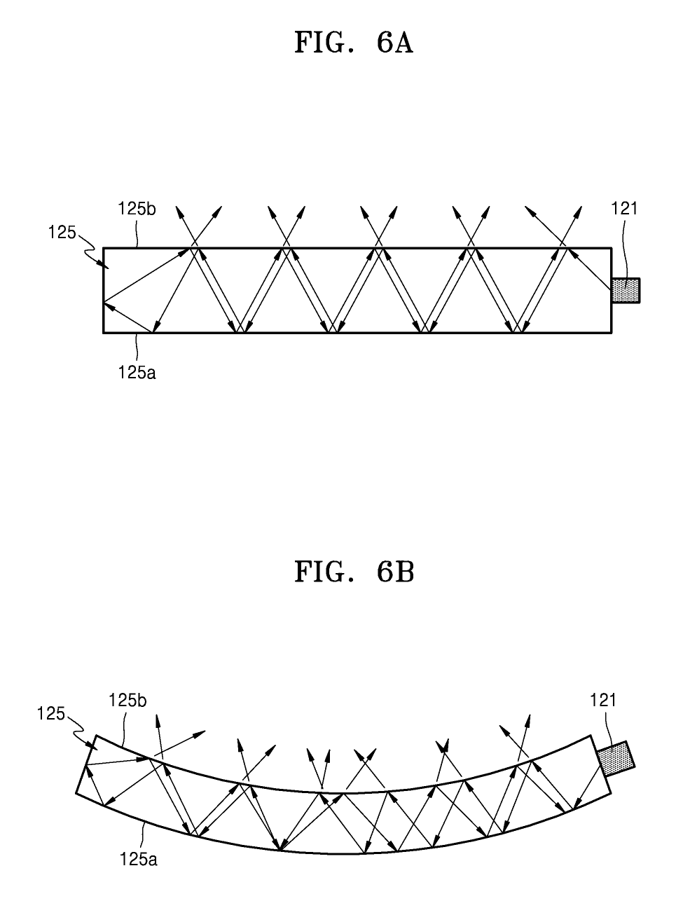

In this case, the light guide 125 may have a reflective surface 125a for reflecting the light incident from the at least one light-emitting device 121 and a translucent surface 125b facing the reflective surface 125a, as shown in FIGS. 6A and 6B. In addition, the light guide 125 may be configured to be bendable so that the apparatus 10 is implemented as a wearable device. FIG. 6A illustrates an unfolded state of the light guide 125, and FIG. 6B illustrates a curved state of the light guide 125.

Referring back to FIG. 2, the pressure sensor 150 of the sensor 100 is configured to acquire the second signal by detecting a pressure change due to the object and may include a plurality of pressure sensors 151.

The plurality of pressure sensors 151 may be arranged to form an array along at least one side of the array arrangement of the plurality of light-receiving devices 131. FIG. 2 shows a case where the plurality of light-receiving devices 131 are arranged in an array along both sides of the array arrangement of the plurality of light-emitting devices 121, and the plurality of pressure sensors 151 are arranged in an array outside the array arrangement of the plurality of light-receiving devices 131. For example, the plurality of pressure sensors 151 may be arranged in an array to one-to-one correspond to the plurality of light-receiving devices 131 at both sides of the photo sensor 110.

A contact-type pressure sensor, e.g., a pressure sensor of a strain gate type, may be used as the plurality of pressure sensors 151 of the pressure sensor 150.

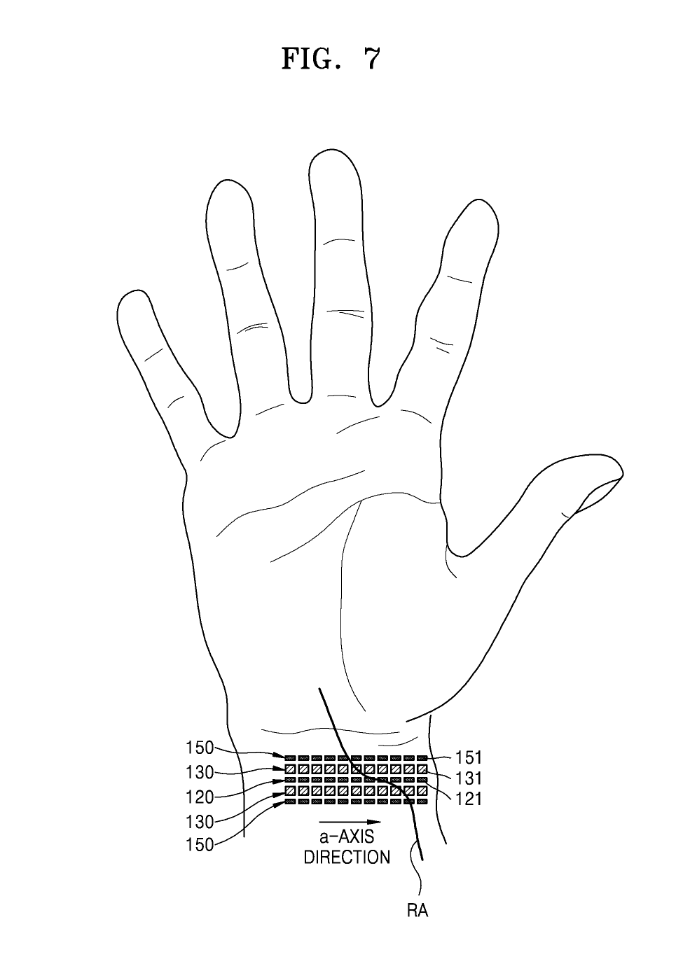

As described above, when the sensor 100 includes the photo sensor 110 and the pressure sensor 150 arranged in an array form, the sensor 100 may be disposed, for example, to be across a blood vessel RA when worn by a user, e.g., worn around a user's wrist, as shown in FIG. 7, and thus, the sensitivity of a signal at which waveform information is extracted from a corresponding a blood vessel may increase without placing much limitation to a wearing state of the apparatus 10.

FIGS. 7, 9, and 15A show examples in which the plurality of light-emitting devices 121 of the light-emitting unit 120 and the plurality of light-receiving devices 131 of the light-receiving unit 130 in the photo sensor 110 have the arrangement as shown in FIGS. 2 and 3. However, the plurality of light-emitting devices 121 of the light-emitting unit 120 and the plurality of light-receiving devices 131 of the light-receiving unit 130 in the photo sensor 110 may have the arrangement as shown in FIG. 4 or 5.

As shown in FIG. 7, when the apparatus 10 is implemented as a wearable device and is worn around a wrist or the like, even when the apparatus 10 turns in an a-axis direction, at least some light-emitting devices 121, at least some light-receiving devices 131, and at least some pressure sensors 151 in the photo sensor 110 and the pressure sensor 150 forming the sensor 100 may be located on a radial artery. Therefore, the apparatus 10 may detect a biometric signal having a higher signal sensitivity.

A pulse waveform of a detected biometric signal may be measured with a different amplitude according to a position along an a-axis, as shown in FIG. 8. For example, a maximum waveform amplitude may be measured at the center of a blood vessel, and a smaller waveform amplitude may be measured at a position farther from the center of the blood vessel. Since the photo sensor 110 and the pressure sensor 150 are arranged in an array form, both the photo sensor 110 and the pressure sensor 150 may exhibit the characteristic of a pulse waveform as shown in FIG. 8. FIG. 8 is a graph illustrating waveforms measured by the photo sensor 110 arranged in an array form and shows an example in which waveform amplitudes are differently measured according to positions along the a-axis direction. In FIG. 8, it is shown that some waveforms are inverted. The inverted waveforms may be corrected by the signal corrector 210 as described above.

As shown in FIG. 8, by arranging the photo sensor 110 and the pressure sensor 150 in an array form, a pulse waveform exhibiting a desired signal sensitivity, e.g., a maximum signal sensitivity, may be obtained. Therefore, a waveform of a biometric signal may be measured regardless of a wearing state of the apparatus 10, and thus convenience of the user may increase, and a wearable device for measuring continuous blood pressure waveforms may be implemented.

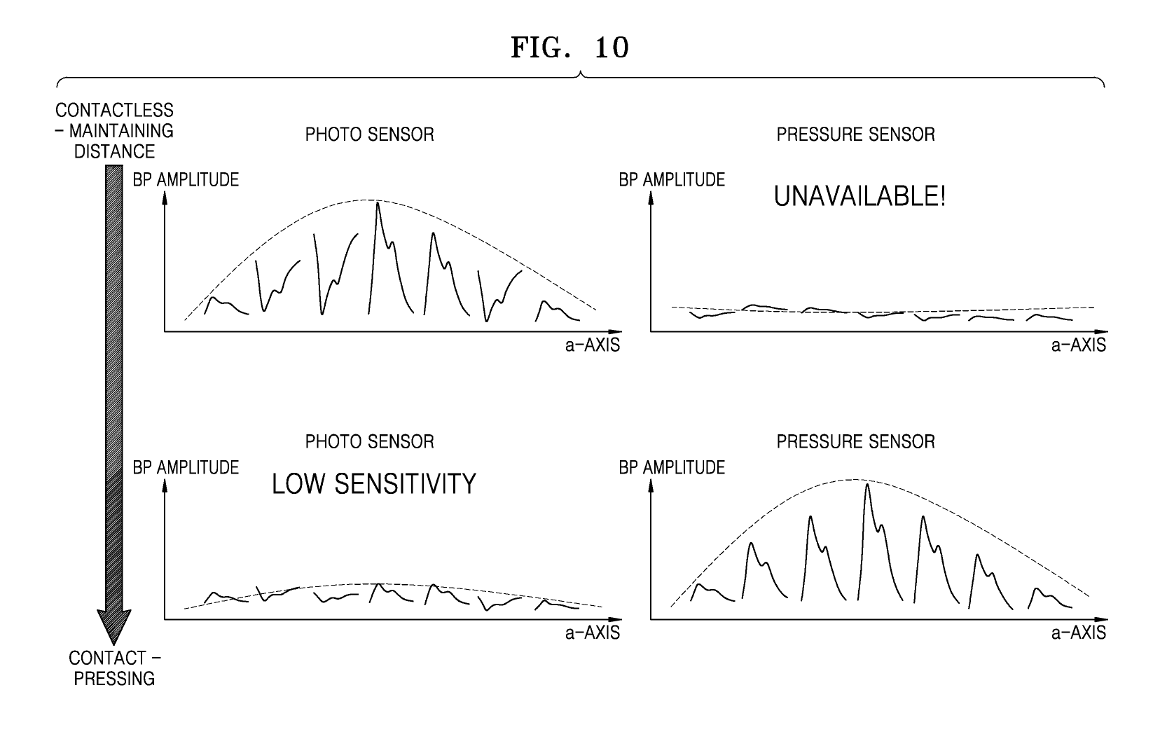

FIG. 9 illustrates an arrangement in which the photo sensor 110 and the pressure sensor 150 are arranged in an array form on a flexible band (or strap) when the apparatus 10 is implemented as a band type or a watch type, according to an exemplary embodiment. FIG. 9 shows a case where the photo sensor 110 includes an array of a plurality of light-emitting devices 121 and has an arrangement of a plurality of light-receiving devices 131 along both sides thereof. However, this is only an example and the exemplary embodiments are not limited thereto. For example the photo sensor 110 may have a structure in which the photo sensor 110 includes an array of a plurality of light-emitting devices 121 spaced apart from each other and has an arrangement of a plurality of light-receiving devices 131 surrounding each light-emitting device 121 as described with reference to FIG. 4 or a structure in which the photo sensor 110 includes the light-emitting unit 120 including at least one light-emitting device 121 and the light guide 125 for guiding light therefrom and has an arrangement of a plurality of light-receiving devices 131 along at least one side of the light-emitting unit 120 as described with reference to FIGS. 5 to 6B may be applied.

FIG. 10 shows graphs illustrating pulse waveforms measured by the photo sensor 110 and the pressure sensor 150 according to contactless and contact states. FIG. 10 shows a case where a laser device is used as the light-emitting device 121 of the photo sensor 110 to measure the pulse waveforms.

Referring to FIG. 10, when the sensor 100 maintains a distance from the object in a contactless state, a pulse waveform is measured with higher signal sensitivity from the photo sensor 110, but a meaningful pulse waveform is not measured from the pressure sensor 150. On the other hand, when the sensor 100 maintains a pressing state of a predetermined level or more in a contact state with the object, the sensitivity of the photo sensor 110 is lowered, and a pulse waveform is measured with higher signal sensitivity from the pressure sensor 150.

As shown in FIG. 10, when pulse waveforms are measured, the measurement waveform of the photo sensor 110 and the measurement waveform of the pressure sensor 150 may differ from each other, and a waveform inversion phenomenon may occur in a portion of the waveform measured in the photo sensor 110.

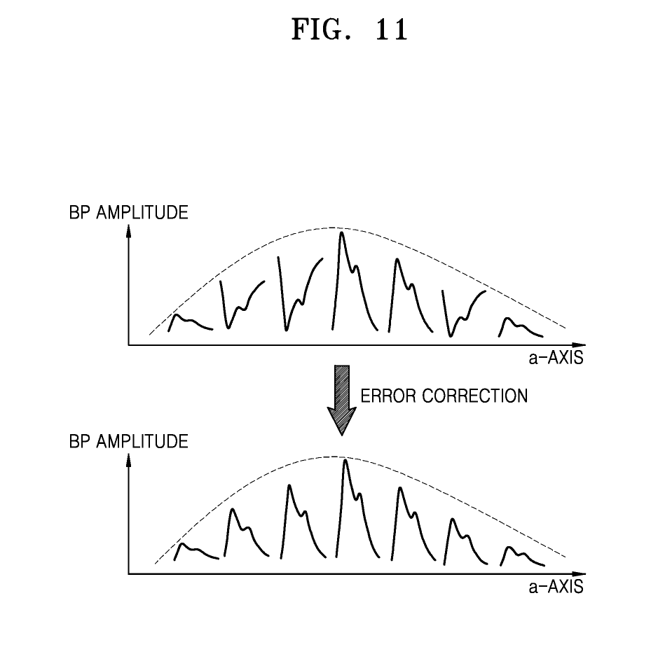

Therefore, the measured waveform of the photo sensor 110 may be corrected to a non-inversion state of waveforms through error correction as shown in FIG. 11.

FIG. 12 shows graphs illustrating comparison of a normal BPW, an inverse BPW, and first-order differential values thereof in a measurement waveform of the photo sensor 110.

As shown in FIG. 12, when the normal BPW and the inverse BPW are first-order differentiated, a maximum absolute value of the first-order differential value thereof has a positive or negative value.

Therefore, through comparison of first differential values of the first signal detected by the photo sensor 110, errors are corrected such that a waveform is inverted again, e.g., when a maximum absolute value of the first-order differential value has a negative value, and thus a pulse waveform of the first signal having various amplitudes of which waveform inversion has been corrected may be obtained.

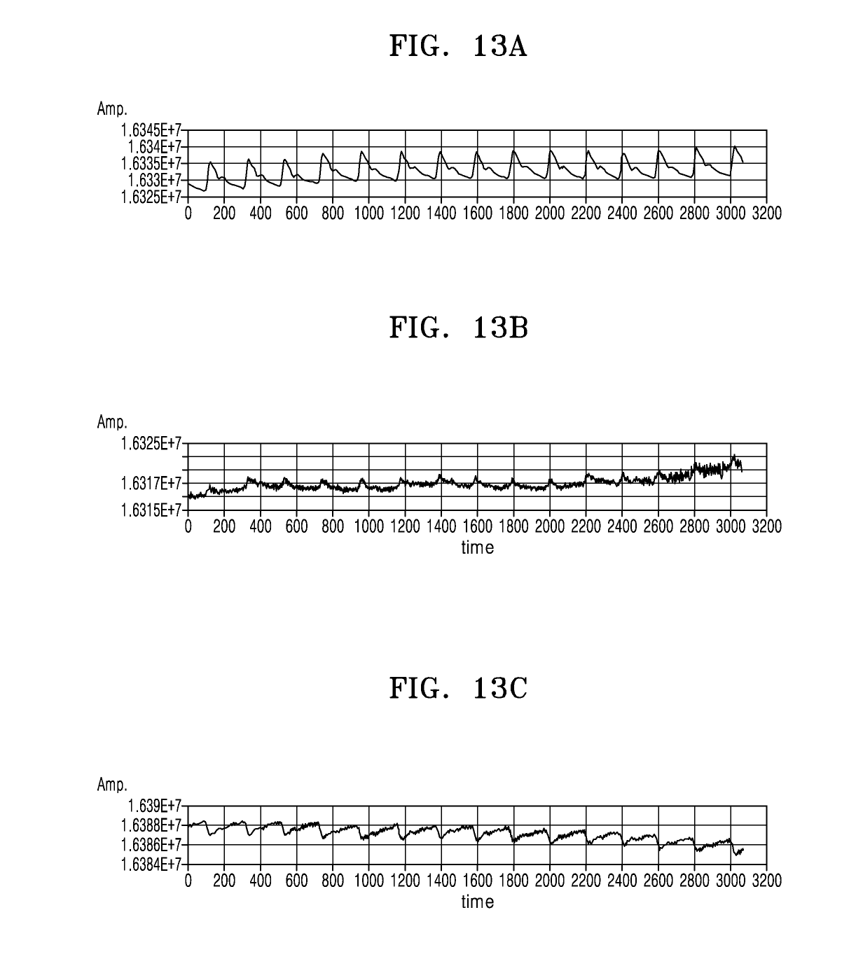

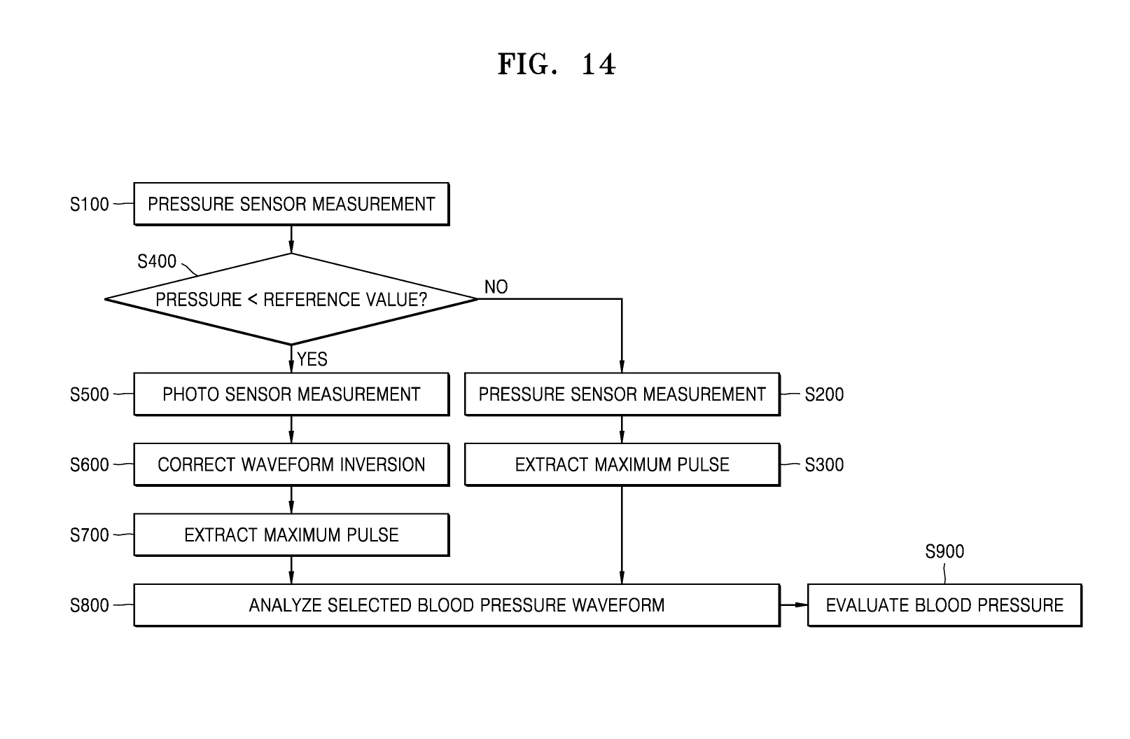

FIGS. 13A to 13C are graphs illustrating various measured waveforms for channels of the photo sensor 110 of the apparatus 10, according to exemplary embodiments. The measurement waveforms of FIGS. 13A to 13C are illustratively obtained through experiments. However, measurement data from the photo sensor 110 of the apparatus 10 is not limited to FIGS. 13A to 13C and may vary according to configurations of the apparatus 10 and measurement conditions.

As shown in FIGS. 13A to 13C, an amplitude of a measured waveform may vary according to a relative position and a distance between the photo sensor 110 of the sensor 100 and the object and a contact pressure between the sensor 100 and the object, and a waveform inversion phenomenon may occur as shown in FIG. 13C. As shown in FIG. 13A, for example, when the sensor 100 maintains a distance from the object in a contactless state, and a relative position between the photo sensor 110 and the object is suitable for measurement, a measured waveform having a higher signal sensitivity may be obtained. As shown in FIG. 13C, when a waveform inversion phenomenon occurs, a measurement waveform may be corrected to a non-inversion state of waveforms through error correction.

As described above, when a pulse waveform having a desired signal sensitivity, e.g., a maximum signal sensitivity, is extracted from measurement waveforms of various amplitudes obtained using the photo sensor 110 and the pressure sensor 150, the pulse waveform having accurate biometric information may be extracted. The signal analyzer 250 may obtain biometric information, e.g., blood pressure information, by analyzing the extracted pulse waveform.

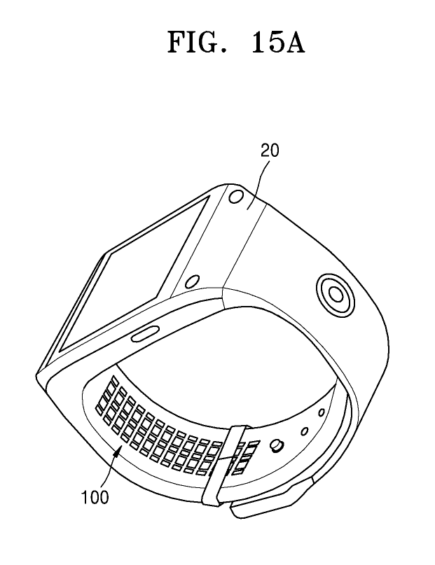

FIG. 14 is a flowchart illustrating a method of detecting biometric information, according to an exemplary embodiment. FIG. 14 illustrates an operation of detecting blood pressure as an example. Even when biometric information other than blood pressure is detected, the biometric information may be detected by the same method as described with reference to FIG. 14. That is, the method of FIG. 14 may be applied to not only detection of blood pressure but also detection of various kinds of biometric information.

Referring to FIG. 14, to detect the biometric information, whether a contact pressure with an object is less than a reference value is determined using the pressure sensor 150 in operations S100 and S400.

When the contact pressure is less than the reference value, the sensor 100 maintains a predetermined distance from the object in a contactless state or is slightly in contact with the object, and thus a biometric signal may be measured using the photo sensor 110 in operation S500. When waveform inversion exists in biometric signal waveforms measured using the photo sensor 110, the waveform inversion is corrected in operation S600. In operation S700, a pulse waveform of a desired signal sensitivity, e.g., a maximum signal sensitivity is extracted from biometric signal waveforms.

When the contact pressure is the reference value or more, the sensor 100 is relatively strongly pressed to the object in a contact state, and thus a biometric signal may be measured using the pressure sensor 150 in operation S200. In operation S300, a pulse waveform of a desired signal sensitivity, e.g., a maximum signal sensitivity may be extracted from pulse waveforms of the biometric signal measured using the pressure sensor 150.

In operations S800 and S900, blood pressure may be evaluated by analyzing a pulse waveform of the maximum signal sensitivity selected from the biometric signal measured from the photo sensor 110 or the pressure sensor 150, e.g., a selected maximum blood pressure waveform.

FIG. 14 shows a case where a contact state is first determined using the pressure sensor 150, and a biometric signal is measured using any one of the photo sensor 110 or the pressure sensor 150. However, the operation of determining a contact state in advance by using the pressure sensor 150 may be omitted. In this case, biometric signals are measured from both the photo sensor 110 and the pressure sensor 150, and biometric information such as blood pressure may be analyzed by extracting a pulse waveform of a desired signal sensitivity, e.g., a maximum pulse waveform, from the measured biometric signals.

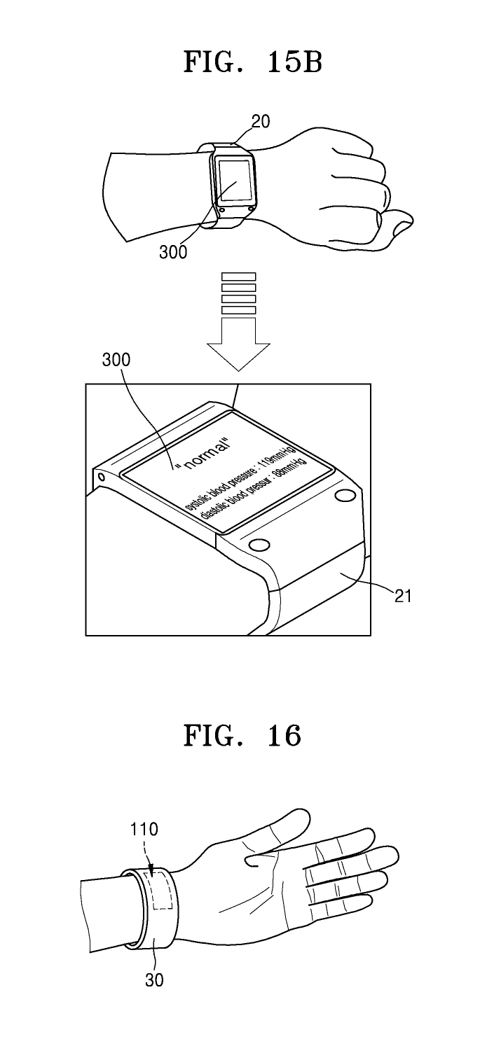

FIG. 15A illustrates a watch-type apparatus 20 for detecting biometric information, according to another exemplary embodiment, and FIG. 15B illustrates the apparatus 20 of FIG. 15A in a using state.

Referring to FIGS. 15A and 15B, the sensor 100 may be provided to a strap portion 21, and biometric information may be obtained by detecting a biometric signal according to a change in the flow of blood flowing through a radial artery.

Biometric information generated by the signal processor 200 may be provided through a display screen of the display 300 of the apparatus 20 that may be worn around a wrist of an object. Information on blood pressure may include, for example, numeric value information of minimum blood pressure and maximum blood pressure of the object, numeric value information of systolic blood pressure and diastolic blood pressure of the object, information on whether a current blood pressure state is normal, blood vessel elasticity information, and the like.

FIG. 16 illustrates a wrist band-type apparatus 30 for detecting biometric information, according to another exemplary embodiment. The wrist band-type apparatus 30 may include the sensor 100 in a band portion thereof.

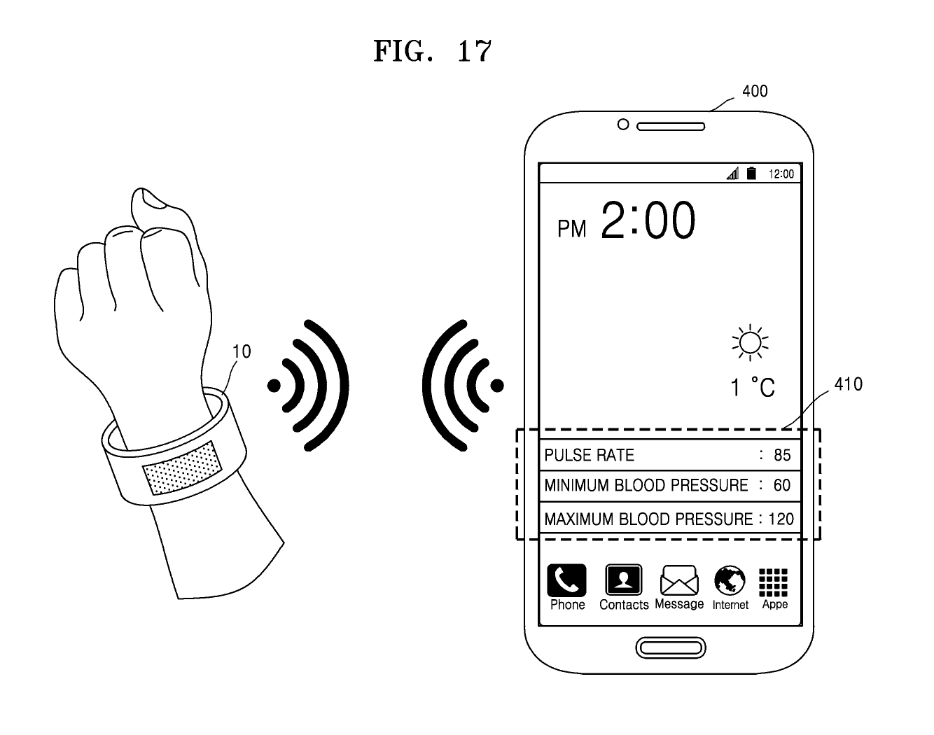

FIG. 17 illustrates a reference diagram for describing a method of providing information on blood pressure when the apparatus 10 is of the wrist band type as shown in FIG. 16.

Referring to FIG. 17, when a wireless communication function such as Bluetooth or Wi-Fi is provided to the apparatus 10, the apparatus 10 may transmit monitored blood pressure information 410 to a smartphone 400 or the like of an object by using the wireless communication function. Accordingly, the object may obtain the blood pressure information 410 through a display screen of the smartphone 400 or the like and/or a display screen of the display 30 of the apparatus 10.

As described above, according to an apparatus and a method for detecting biometric information according to the one or more of the above exemplary embodiments, a sensor includes a photo sensor and a pressure sensor arranged in an array form, waveform inversion is corrected when the waveform inversion exists in a measurement waveform of a photo sensor, and thus a pulse waveform of the maximum sensitivity may be extracted in a contactless or contact manner, and biometric information may be continuously acquired.

It should be understood that exemplary embodiments described herein should be considered in a descriptive sense only and not for purposes of limitation. Descriptions of features or aspects within each exemplary embodiment should typically be considered as available for other similar features or aspects in other exemplary embodiments.

At least one of the components, elements or units represented by a block as illustrated in FIG. 1 may be embodied as various numbers of hardware, software and/or firmware structures that execute respective functions described above, according to an exemplary embodiment. For example, at least one of these components, elements or units may use a direct circuit structure, such as a memory, processing, logic, a look-up table, etc. that may execute the respective functions through controls of one or more microprocessors or other control apparatuses. Also, at least one of these components, elements or units may be specifically embodied by a module, a program, or a part of code, which contains one or more executable instructions for performing specified logic functions. Also, at least one of these components, elements or units may further include a processor such as a central processing unit (CPU) that performs the respective functions, a microprocessor, or the like. Further, although a bus is not illustrated in the above block diagrams, communication between the components, elements or units may be performed through the bus. Functional aspects of the above exemplary embodiments may be implemented in algorithms that execute on one or more processors. Furthermore, the components, elements or units represented by a block or processing steps may employ any number of related art techniques for electronics configuration, signal processing and/or control, data processing and the like.

Although a few embodiments have been shown and described, it would be appreciated by those skilled in the art that changes may be made in the exemplary embodiments without departing from the principles and spirit of the disclosure, the scope of which is defined in the claims and their equivalents.

What is claimed is:

1. An apparatus for detecting biometric information, the apparatus comprising:
a photo sensor configured to acquire a first signal by detecting a change in an optical signal with respect to an object;
a pressure sensor configured to acquire a second signal by detecting a change of a pressure with respect to the object;
a signal corrector configured to correct a waveform inversion that occurs in the first signal;
a signal extractor configured to extract a waveform signal from at least one from among the first signal and the second signal based on signal sensitivities of the first and second signals; and
a signal analyzer configured to analyze biometric information from the extracted waveform signal.

2. The apparatus of claim 1, wherein the photo sensor is further configured to acquire the first signal in a contactless state with respect to the object or when a contact pressure with respect to the object is less than a reference value, and
the pressure sensor is configured to acquire the second signal in a contact state with respect to the object.

3. The apparatus of claim 1, wherein the photo sensor comprises:
a light-emitting unit comprising at least one light-emitting device, and is configured to emit light; and
a light-receiving unit comprising at least one light-receiving device configured to detect the change in the optical signal.

4. The apparatus of claim 3, wherein the light-emitting unit comprises a plurality of light-emitting devices that are arranged in at least one line or the light-emitting unit comprises a light guide configured to guide light incident from the at least one light-emitting device.

5. The apparatus of claim 4, wherein the light-receiving unit comprises a plurality of light-receiving devices, the plurality of light-receiving devices being arranged in an array along at least one side of the light-emitting unit or arranged between the plurality of light-emitting devices such that the plurality of light-receiving devices surround each of the plurality of light-emitting devices.

6. The apparatus of claim 4, wherein the light guide has a reflective surface, on which the light incident from the at least one light-emitting device is reflected, and a translucent surface that faces the reflective surface.

7. The apparatus of claim 6, wherein the light guide is bendable.

8. The apparatus of claim 3, wherein the photo sensor has a structure in which a unit of a light-emitting device and a pair of light-receiving devices located at both sides of the light-emitting device is arranged in an array or the photo sensor has a structure in which a unit of the light-emitting device and a plurality of light-receiving devices surrounding the light-emitting device is arranged in an array.

9. The apparatus of claim 8, wherein the pressure sensor comprises a plurality of pressure sensors that are arranged in an array at both sides of the photo sensor in a one-to-one correspondence with the plurality of light-receiving devices.

10. The apparatus of claim 5, wherein the pressure sensor comprises a plurality of pressure sensors that are arranged in an array along at least one side of the array of the plurality of light-receiving devices.

11. The apparatus of claim 1, wherein the photo sensor comprises:
a light-emitting unit comprising at least one light-emitting device, and configured to emit light; and
a light-receiving unit comprising a plurality of light-receiving devices that are arranged in an array along at least one side of the light-emitting unit, and
the pressure sensor comprises a plurality of pressure sensors arranged along the array of the plurality of light-receiving devices.

12. The apparatus of claim 11, wherein the photo sensor comprises a plurality of light-emitting devices that are arranged to form at least one line or the photo sensor comprises a light guide configured to guide light from the at least one light-emitting device.

13. The apparatus of claim 12, wherein the light guide is bendable.

14. The apparatus of claim 3, wherein the light-emitting device comprises a laser device.

15. The apparatus of claim 1, wherein the signal corrector is further configured to correct the waveform inversion that occurs in the first signal based on comparison between first-order differential values of the first signal.

16. The apparatus of claim 1, wherein the biometric information comprises information about blood pressure of the object.

17. A method of detecting biometric information, the method comprising:
acquiring a first signal by detecting a change in an optical signal with respect to an object;
acquiring a second signal by detecting a change of a pressure with respect to the object;
correcting waveform inversion that occurs in the first signal;
extracting a waveform signal from at least one from among the first signal and the second signal based on signal sensitivities of the first and second signals; and
analyzing biometric information from the extracted waveform signal.

18. The method of claim 17, wherein the extracting comprises, when a contact pressure with respect to the object is less than a reference value, extracting the waveform signal from the first signal.

19. The method of claim 17, wherein the waveform inversion is corrected based on comparison between first-order differential values of the first signal.

20. The method of claim 17, wherein the biometric information comprises information about blood pressure of the object.
